(19) United States
(12) Patent Application Publication
(10) Pub. No.: US 2025/0389722 A1
(43) Pub. Date: Dec. 25, 2025

(54) HYBRID METAL NANOPARTICLES FOR ULTRAFAST DETECTION OF PANCREATIC DUCTAL ADENOCARCINOMA

(71) Applicant: Georgia Tech Research Corporation, Atlanta, GA (US)

(72) Inventors: Nasrin Hooshmand, Atlanta, GA (US); Lasya Maganti, Atlanta, GA (US); Ashkan Zandi, Atlanta, GA (US)

(21) Appl. No.: 19/244,933

(22) Filed: Jun. 20, 2025

Related U.S. Application Data
(60) Provisional application No. 63/662,476, filed on Jun. 21, 2024.

(51) Int. Cl. G01N 33/574; G01N 33/543

(52) U.S. Cl. CPC G01N 33/57438; G01N 33/54373; G01N 33/57473; G01N 33/57488; G01N 33/57492

(57) ABSTRACT

The present disclosure is directed towards methods for evaluating the presence of a cancer within a subject. Such methods include obtaining a biological sample from the subject, identifying a biological component in the biological sample, wherein the identity of the biological component is indicative of the presence or absence of a cancer. The methods disclosed herein may further include quantifying the amount of the biological component in the biological sample, wherein the quantity of the biological component is indicative of the presence or absence of a cancer. The present disclosure is also directed to systems for evaluating the presence of cancer within a subject, such device including a biological component detector comprising nanowires functionalized with nanoparticles and antibodies.

### CROSS-REFERENCE TO RELATED APPLICATION

This application claims the benefit of U.S. Provisional Application No. 63/662,476 filed on Jun. 21, 2024, the entire contents of which are incorporated by reference herein.

### BACKGROUND OF THE INVENTION

Pancreatic cancer is a highly aggressive form of cancer that affects the pancreas. It is characterized by the uncontrolled growth of cells in the pancreas and can be challenging to detect in its early stages. Pancreatic cancer is often diagnosed at an advanced stage, which makes it one of the deadliest forms of cancer. Pancreatic Cancer has one of the lowest survival rates among patients once diagnosed due to a variety of reasons such as genetic heterogeneity and lack of treatments. One critical reason for high mortality is the lack of preventative measures and early diagnosis in patients.

The present invention looks to solve diagnosis issues by testing patients for biomarkers that allow for diagnosis of pancreatic adenocarcinoma (PDAC), which accounts for approximately 95% of cases of pancreatic cancer.

FIG. 1A is an anatomical illustration of the pancreas. This schematic depicts the location and depth of the pancreas within the human body, emphasizing its relative position behind the stomach and in close proximity to other vital organs such as the small intestine, spleen, liver, and major blood vessels. The illustration highlights the pancreas' deep-seated placement, underscoring the complexities and challenges associated with accessing the organ for diagnostic and therapeutic procedures. All sizes have been exaggerated for demonstration purposes. The inset of FIG. 1A provides a focused schematic of the pancreas, gallbladder, and common bile duct, illustrating the pathway of digestive enzymes from the pancreas to the duodenum. It highlights the integral role of the pancreas in digestion, showcasing the release and flow of enzymes essential for the breakdown of food in the gastrointestinal tract.

FIG. 1B is a schematic representation that provides a comprehensive view of the pancreas afflicted by a pancreatic tumor, highlighting the involvement of adjacent lymph nodes and the metastasis to the liver. FIG. 1B details the pancreatic secretion within the duodenum. Additionally, the diagram underscores the presence of pancreatic cancer-derived exosomes.

### SUMMARY OF THE INVENTION

Various aspects of the disclosure are directed to biological component detectors for the ultrafast detection of cancer markers, such as pancreatic cancer or more specifically Pancreatic Ductal Adenocarcinoma (PDAC) markers using hybrid metal nanoparticles. Such detectors integrate hybrid nanoparticles, comprising or consisting of noble metals such as gold and silver, with semiconductor nanowires made of, for example, silicon or zinc oxide (ZnO). The nanoparticles exhibit localized surface plasmon resonance (LSPR), which enhances their optical absorption and scattering properties for detecting biomarkers associated with pancreatic cancer.

Biological component detectors according to various aspects of the disclosure operate by utilizing a field-effect transistor (FET) design, where antibody-functionalized semiconductor nanowires, such as ZnO nanowires, form a channel that modulates electrical conductivity upon interaction with target molecules. In some instances, the target molecules are exosomes from cancer cells, such as pancreatic cancer or PDAC cells. PDAC exosomes carry markers like Glypican-1 (GPC-1) and macrophage migration inhibitory factor (MIF), which, when bound to antibodies on the semiconductor nanowires, alter the local dielectric constant affecting the LSPR properties of the nanoparticles on the semiconductor nanowires.

Biological component detectors according to various aspects of the disclosure are highly sensitive and specific due to the large surface area and electronic properties of the functionalized semiconductor nanowires, combined with the unique optical features of the plasmonic nanoparticles. The biological component detectors described herein are designed to enable real-time, rapid detection of pancreatic cancer biomarkers, providing a significant improvement over conventional biosensors in terms of sensitivity, specificity, and time efficiency, and will greatly enhance early-stage diagnosis and monitoring of pancreatic cancer.

Biological component detectors according to various aspects of the disclosure offer several advantages over existing technologies, which typically include various imaging techniques, traditional biopsy methods, and standard biochemical assays.

One advantage of biological component detectors according to various aspects of the disclosure over existing technologies is enhanced sensitivity and specificity. The use of noble metal nanoparticles like gold and silver, which exhibit localized surface plasmon resonance (LSPR), significantly enhances the optical properties of the biological component detectors. That allows for the detection of biological components at much lower concentrations than many current methods can achieve. In some instances, the biological component detectors bind to PDAC biomarkers. In some instances, the PDAC biomarkers are Glypican 1 (GPC-1) and macrophage migration inhibitory factor (MIF).

Another advantage of biological component detectors according to various aspects of the disclosure over existing technologies is rapid detection. One of the standout features of these biological component detectors according to the disclosure is their ability to provide results quickly. Rapid detection is crucial for early diagnosis and timely treatment initiation, which are critical factors in improving outcomes for cancer patients.

Another advantage of biological component detectors according to various aspects of the disclosure over existing technologies is minimal sample preparation. The biological component detectors are designed to work with minimal sample preparation, reducing the overall time and cost associated with the diagnostic process. That contrasts with many traditional methods that require extensive sample preparation and processing.

Another advantage of biological component detectors according to various aspects of the disclosure over existing technologies is real-time monitoring. The biological component detectors enable real-time monitoring of the interaction between biomarkers and the device, which can be crucial for assessing disease progression or response to treatment. That is a significant improvement over techniques like MRI or CT scans, which provide only snapshots in time.

Another advantage of biological component detectors according to various aspects of the disclosure over existing technologies is cost savings. By simplifying the detection process and reducing reliance on expensive imaging equipment and highly specialized laboratory facilities, biological component detectors according to the disclosure may lower the costs of cancer diagnostics.

Another advantage of biological component detectors according to various aspects of the disclosure over existing technologies is portability and Point-of-Care (POC) application. The potential for miniaturization and portability means that the biological component detectors may be used in a point-of-care setting. That is particularly beneficial in low-resource environments or in situations where immediate results are needed, such as during surgical procedures.

Another advantage of biological component detectors according to various aspects of the disclosure over existing technologies is reduced invasiveness. Unlike a biopsy, which is invasive and can be painful or even potentially life-threatening for the patient, biological component detectors according to the present disclosure are envisioned to require only a small biological sample, such as blood or exosomes extracted from blood, making the process less invasive.

### BRIEF DESCRIPTION OF DRAWINGS

FIG. 1A is an anatomical illustration of the pancreas depicting the location and depth of the pancreas within the human body.

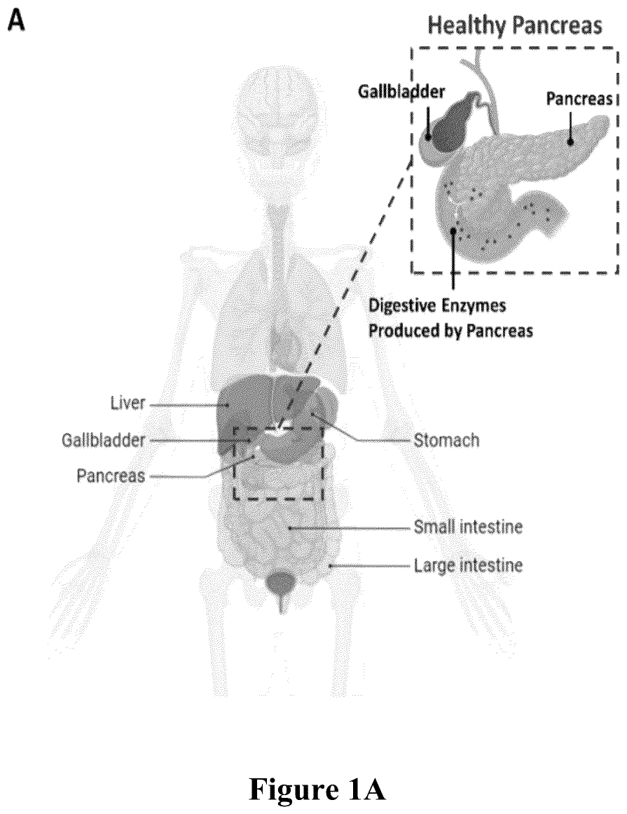

FIG. 1B is a schematic representation of a pancreas afflicted by a pancreatic tumor and surrounding metastasis.

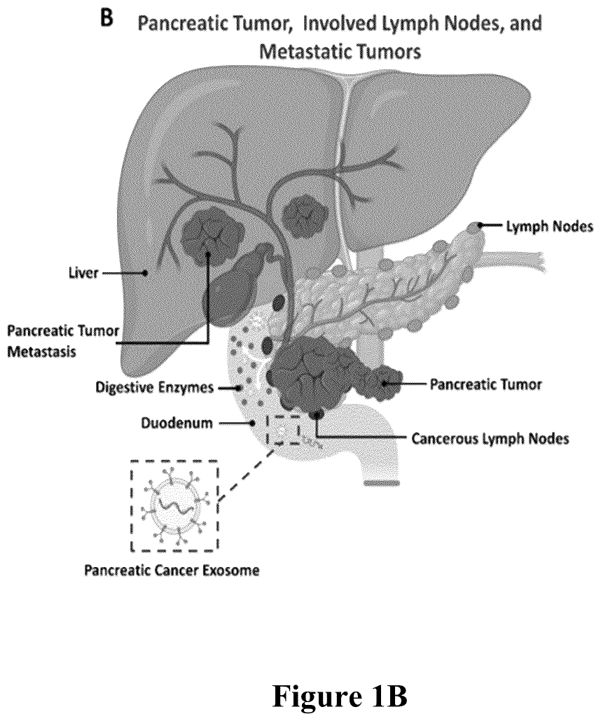

FIG. 2 is a schematic illustration of hybrid metal nanowires with nanoparticles and antibodies adhered to the nanowire for detection of biological components.

FIG. 3 is a schematic illustration of an exemplary biological component detector according to various aspects of the disclosure.

FIG. 4 is a schematic illustration of the vibrations of nanoparticles upon a shift in refractive index.

### DETAILED DESCRIPTION OF THE INVENTION

Various aspects of the disclosure pertain to methods of identifying biological components from a biological sample. The biological components to be identified and optionally quantified may serve as indicators of a disease state of a subject from which the biological sample originated. The devices and methods described herein may be applied to various types of biological samples. In accordance with various aspects of the disclosure, the devices and method described herein have been found particularly useful for identification and quantification of biological components of blood, stool and tissue samples.

Various types of biological components in biological samples can be identified and quantified using the devices and methods described herein such as, but not limited to, nucleic acids (for example, DNA and RNA), peptides, proteins, cells, exosomes, antigens, viruses, and bacteria. In accordance with various aspects of the disclosure, the devices and methods described herein have been found particularly useful for the identification and quantification of biological components of biological samples, such as blood, stool and tissue samples, that are indicative of various forms of cancer. In some instances, the biological sample is a bodily fluid from a subject, such as saliva, blood, mucus, cerebrospinal fluid (CSF), amniotic fluid or urine. In some instances, the biological sample is extracted from a tissue sample, such as a tissue biopsy. For biological component detection, biological samples generally undergo some form of processing, such as liquification and/or homogenization, for breakdown of the biological sample prior to testing using the devices and methods described herein.

More particularly, the devices and methods described herein have been particularly useful for the identification and quantification of exosomes and antigens that are indicative of various forms of cancers, including forms of pancreatic cancers such as exocrine pancreatic cancer and endocrine pancreatic cancer. In some instances, the exocrine cancer is pancreatic ductal adenocarcinoma (PDAC).

Generally, devices for quantifying biological components from a biological sample comprise a biological component detector according to various aspects of the disclosure.

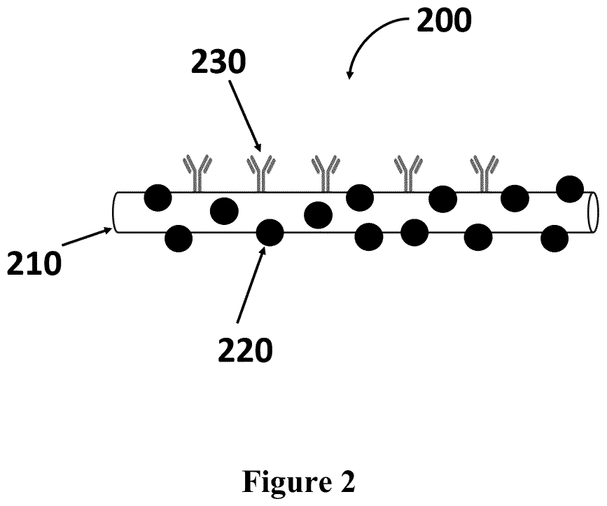

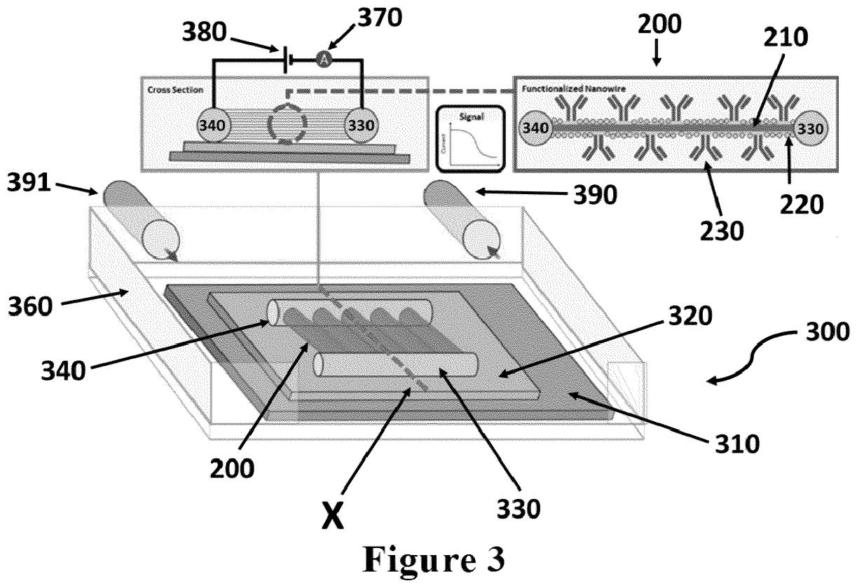

FIG. 2 is a schematic illustration of a functionalized semiconductor nanowire 200 to be used in a biological component detector as described in, for example, FIG. 3. As illustrated in FIG. 2, functionalized semiconductor nanowires 200 according to various aspects of the disclosure may generally include a semiconductor nanowire 210 and a plurality of nanoparticles 220 and antibodies 230 located on the surface of the semiconductor nanowire 210. In some instances, the semiconductor nanowire 210 is made of copper, zinc, gold, or silver, silicon, or oxides or alloys comprising, consisting essentially of, or consisting of the same. In some instances, the use of zinc oxide or silicon as the semiconductor nanowire 210 composition is preferred. As illustrated in FIG. 2, the semiconductor nanowire 210 includes a plurality of nanoparticles 220 adhered to the semiconductor nanowire 210 surface. The nanoparticles 220 can comprise, consist essentially of, or consist of gold, silver, ruthenium, rhodium, palladium, osmium, iridium, platinum, or alloys thereof. The semiconductor nanowire 210 further comprises antibodies 230 bound to its surface. The antibodies 230 exhibit a binding affinity to biological components (e.g., nucleic acids (e.g., DNA and RNA), peptides, proteins, cells exosomes, antigens, viruses, and bacteria) for detection and optionally quantification of the same. Examples of antibodies that can be used as antibodies 230 include a variety of cancer markers, including pancreatic cancer parkers, and biological species including, but not limited to, GPC-1 antibodies, MIF antibodies, CEA antibodies, CA19-9 antibodies, EpCAM antibodies, CD24 antibodies, CD44 antibodies, CD62 antibodies, CD81 antibodies, Kras antibodies, and CD9 antibodies. Such antibodies bind to various receptors on cancer exosome surfaces such as GPC-1, MIF, CEA, CA19-9, EpCAM, CD24, CD44, CD62, CD81, Kras and CD9. In some instances, the use of GPC-1 or MIF antibodies as the antibodies 230 is preferred. In practice, biological components of biological samples will bind to the antibodies 230 on the functionalized semiconductor nanowire 200, allowing for identification and quantification of the biological components.

In some instances, functionalized semiconductors nanowires 200 according to various aspects of the disclosure can have lengths ranging from about 1 to about 10 μm and diameters ranging from about 30 to about 100 nanometers. In some instances, nanoparticles 220 of functionalized semiconductor nanowires 200 according to various aspects of the disclosure can have average sizes and/or diameters ranging from about 10 to about 30 nanometers. In some instances, the nanoparticles 220 can be grown on the semiconductor nanowires 210 via the in situ reduction of one or more suitable metal-containing salts (for example, gold, silver, ruthenium, rhodium, palladium, osmium, iridium, and/or platinum salts. In particular example, the growth of gold nanoparticles on semiconductor nanowires 210 via the in situ reduction of HAuCl₄ using NaBH₄ as a reducing agent. In another particular example, the growth of silver nanoparticles on semiconductor nanowires 210 via the in situ reduction of AgNO₃ using NaBH₄ or sodium citrate as a reducing agent.

In some instances, the antibodies 230 are bound to the semiconductor nanowires 210 by weak intermolecular bonding interactions, or physisorption. In some instances, the antibodies 230 are bound to the semiconductor nanowires 210 by covalent bonding interactions, or chemisorption. In some instances, it is preferred that the antibodies 230 are covalently bound to the semiconductor nanowires 210. The method of covalent bonding of the antibodies 230 to the semiconductor nanowires 210 is not particularly limited and ma be guided by the nature of reactive chemical functionalities of the antibodies 230 themselves. For example, antibodies containing primary amine groups (e.g., on lysine residues) can be covalently attached to the surface of the semiconductor nanowires 210 using linkers such as APTES and glutaraldehyde. Such a reaction methodology forms imine (Schiff base) bonds between the antibody 230 and the semiconductor nanowires 210.

FIG. 3 is a schematic illustration of an exemplary biological component detector 300, specifically a Field Effect Transistor (FET) configured as a liquid gate transistor, according to various aspects of the disclosure. As illustrated in FIG. 3, the base of the biological component detector 300 is a substrate layer 310. The substrate layer 310 can be made up of, for example, zinc oxide, silicon oxide, or indium tin oxide. On top of the substrate layer 310 is a semiconductor oxide layer 320 which can be made of one or more semiconductor oxide materials. Examples of semiconductor oxide materials for the semiconductor oxide layer 320 include, but are not limited to, zinc oxide, titanium oxide, cesium oxide, magnesium oxide, selenium oxide, or zirconium oxide. Disposed on the semiconductor oxide layer 320 are a source electrode 330 and a drain electrode 340. The source electrode 330 and drain electrode 340 can be made of any suitable metal or metal alloy commonly used as electrodes in FETs. In some instances, the source electrode 330 and drain electrode 340 are made of gold or silver. The source electrode 330 is connected to a voltage source 370, such as, for example, a battery. The drain electrode 340 is connected to a drain source 380. Functionalized semiconductor nanowires, such functionalized semiconductor nanowires 200 as described in FIG. 2, electrically couple the source electrode 330 and the drain electrode 340. The antibodies 230 bound to the functionalized semiconductor nanowires 200 are used to bind to biological components of biological samples. In some instances, the antibodies 230 bind to cancer markers. In some instances, the antibodies bind to pancreatic cancer markers such as GPC-1, MIF, CEA, CA19-9, EpCAM, CD24, CD44, CD62, CD81, and CD9.

As illustrated in FIG. 3, a microfluidic chamber 360 encases the biological component detector 300. The microfluidic chamber 360 comprises an inlet 391 and an outlet 390 for delivery and circulation of a biological sample to the biological component detector 300. The biological sample comprising biological components enters microfluidic chamber 360 via application of pressure or transduction. Prior to entry into the microfluidic chamber 360, the biological sample may undergo pre-processing to facilitate detection of the target biological component(s) to be detected. Examples of pre-processing can include, but are not limited to, liquification, homogenization, ultra-centrifugation, filtration, and so on.

Also shown in FIG. 3 is a cross-sectional illustration, taken along plane X, showing the source electrode 330, the drain electrode 340 and a plurality of functionalized semiconductor nanowires, such functionalized semiconductor nanowires 200 as described in FIG. 2, electrically coupling the source electrode 330 and the drain electrode 340. Also shown in FIG. 3 is a cross-sectional illustration showing the source electrode 330, the drain electrode 340 and single one of a plurality of functionalized semiconductor nanowires, such functionalized semiconductor nanowires 200 as described in FIG. 2, electrically coupling the source electrode 330 and the drain electrode 340.

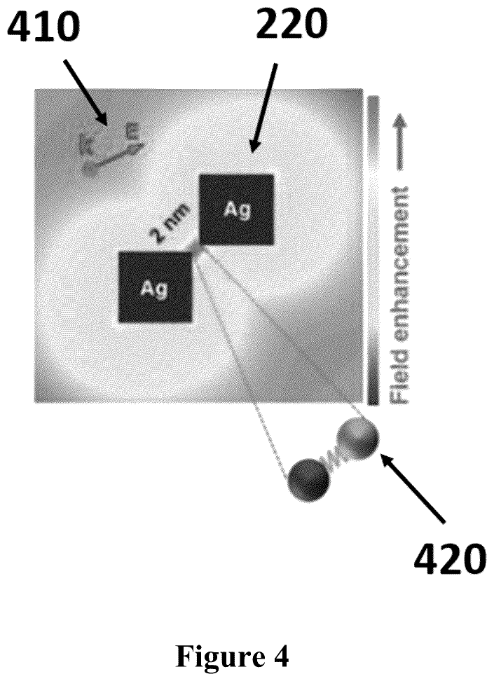

For detection of biological components, a biological sample flows through the microfluidic chamber 360 and over the functionalized semiconductor nanowires 200. The biological components of the biological sample (such as for example nucleic acids (for example, DNA and RNA), peptides, proteins, cells, exosomes, antigens, viruses, and bacteria) bind to the antibodies 230 on the functionalized semiconductor nanowires 200. The binding of these biological components to the antibodies 230 on the functionalized semiconductor nanowires 200 causes a shift in the localized surface plasmon resonance (LSPR) of the nanoparticles 220 on the nanowire. LSPR is a phenomenon where the electrons in the nanoparticles oscillate collectively to incident electromagnetic radiation typically in the visible range of the electromagnetic spectrum. As illustrated in FIG. 4, two nanoparticles 220 in close proximity to each other create a vibration response 420 across a dipolar plane 410. The resonance results in strong optical absorption and scattering properties that are highly sensitive to changes in the particle environment, making them valuable for biosensing applications. To measure the LSPR, a baseline measurement is taken before the biological sample is put into the inlet 391 of the microfluidic chamber 360. The baseline measurement serves as a reference point to capture the optical characteristics of the nanoparticles 220 in their unbound state. Binding of biological components to the antibodies 230 on the functionalized semiconductor nanowire 200 causes a shift of the refractive index wherein a change in the refractive index causes a change in the LSPR of the environment. This shift in LSPR can be measured using optical spectroscopy techniques such as UV-Vis spectroscopy or surface plasmon spectroscopy. This measurement technique provides a sensitive, label-free method for detection without relying on electrical conduction changes. Noble metal nanoparticles, such as gold and silver, provide strong surface plasmon resonance (SPR) effects, which can amplify the signal for better sensitivity. Semiconductor nanowires, on the other hand, offer high surface-to-volume ratios and excellent electronic properties, which can further improve the detection efficiency and specificity. By binding antibodies to both materials, as in functionalized semiconductor nanowires 200, biological detection systems using the same can achieve a synergistic effect, combining the advantages of each nanomaterial to enhance overall performance.

According to various aspects of the disclosure, hybrid nanowire/nanoparticle-based biological component detectors as described herein offer several advantages over existing methods and devices. Those include ultrafast detection, high sensitivity and specificity, lower limit of detection (LOD), and reduced sample preparation and handling. The use of hybrid nanowires/nanoparticles enhances the sensing capabilities compared to bare metallic nanoparticles or semiconducting nanowires.

Biological component detectors according to various aspects of the disclosure have significant commercial potential in the fields of medical diagnostics, pharmaceuticals, and biotechnology. Biological component detectors according to various aspects of the disclosure can be employed for early-stage diagnosis of pancreatic cancer by detecting specific biomarkers in patient samples. Additionally, biological component detectors according to various aspects of the disclosure may find applications in research laboratories, clinical settings, and industries focused on healthcare and diagnostics. The ability of biological component detectors, according to various aspects of the disclosure, to achieve rapid and sensitive detection makes them a valuable tool in a wide range of applications.

For example, biological component detectors according to various aspects of the disclosure will be valuable in the field of medical diagnostics and as research tools. Biological component detectors according to the disclosure will enhance biomedical research, allowing scientists to study cancer progression, treatment efficacy, and the biology of various forms of cancer including PDAC. Biological component detectors disclosed herein can be used in both research settings to quantitatively analyze cancer biomarkers, to accelerate research and development in oncology. Biological component detectors disclosed herein can be used clinical settings for early and rapid detection and monitoring of cancers, such as pancreatic cancers. In clinical settings, the ability of biological component detectors disclosed herein to rapidly cancer biomarkers, such as PDAC biomarkers, from patient samples makes them valuable tools for oncologists and pathologists, allowing for improved patient outcomes through earlier intervention. Furthermore, biological component detectors according to the disclosure are relatively small and compact, which allow for rapid analyses suitable for point-of-care applications, allowing for quick, on-site health assessments, which is particularly useful in remote or underserved areas.

Also for example, biological component detectors according to various aspects of the disclosure will be valuable in the field of drug development. Biological component detectors according to the disclosure will provide pharmaceuticals developers the ability to efficiently evaluate the therapeutic efficacy of new drugs against various lines of cancers, including lines of pancreatic cancers. More specifically, biological component detectors according to the disclosure can be used to monitor the biological responses of cancer cells to treatments with new drugs in in vitro and in vivo preclinical and clinical trials.

Also for example, biological component detectors according to various aspects of the disclosure will be valuable in the research and development personalized medicines. Specifically, biological component detectors according to various aspects of the disclosure can be used as tools for monitoring disease progression and biological responses to treatment regimens. In some instances, the biological component detectors may facilitate personalized treatment regimens for cancer patients, aligning with the broader trend towards personalized medicine in healthcare.

Also for example, biological component detectors according to various aspects of the disclosure will be valuable in veterinary medicine. While this disclosure is primarily directed to the use of biological component detectors according to various aspects of the disclosure for humans, they may equally be applied as medical diagnostics, research tools, clinical tools, and drug development and personalized medicine tools for animals to be treated in veterinary settings.

The detection of small biological molecules is important in, among other applications, environmental analysis, disease diagnosis, food quality control, and drug discovery.¹ Molecules or analytes can be detected by employing biological component detectors, as described herein, as analytical devices which use biologically sensitive molecules or bioreceptors (such as antibodies 230) for the detection of biological components of interest. In some instances, recognition of the analyte by bioreceptors according to various aspects of the disclosure causes a physiochemical change in a transducer (such as nanoparticles 220) to which the bioreceptor is a component. Based on the physiochemical change that happened in the transducer biological component detectors are classified into optical, electrochemical, mass-based, and piezoelectric sensors. In some instances, if a nanoparticle is employed as the transducer, biological component detectors according to the disclosure can be referred to as biosensors. Such biosensors resolve many of the issues related to conventional biosensors such as time consumption, low sensitivity and specificity, high cost, significant sample preparation, and intensive sample handling.² The higher sensitivity and specificity of the biosensors described herein can be attributed to their greater surface to volume ratios and interesting physicochemical properties compared to their bulk counterparts. With that background, making use of hybrid metal nanoparticle semiconducting nanowires (such as functionalized semiconductor nanowires 200) for detecting the biomarkers of pancreatic cancer would be beneficial for patients. Noble metal nanoparticles (for example, gold, silver, ruthenium, rhodium, palladium, osmium, iridium, platinum, or alloys thereof) exhibit strong optical absorption and scattering within the visible range of electromagnetic radiation, attributed to a phenomenon known as localized surface plasmon resonance (LSPR). Such nanoparticles are promising candidates for applications in vivo and within intracellular environments, and their optical and electrical properties can be exploited to increase the sensitivity and specificity of detection and to achieve a lower limit of detection (LOD). Plasmonic metal nanoparticles, especially those made of gold and silver, offer significant promise to detect substances rapidly and down to the single-molecule level. Plasmonic light produced from gold or silver nanoparticles is sensitive to their environment and separation distance.^{3,4} Hybrid nanoparticles have been shown to have even greater improvements towards the sensing compared to bare metallic nanoparticles or semiconducting nanowires.

Hybrid nanostructures can be synthesized by depositing plasmonic nanoparticles on, for example, Si or ZnO nanowires. Hybrid nanoparticle thin films can be used to design electrical devices for the nano-biosensing of exosomes related to PDAC (see FIG. 2). Biological component detector designs according to various aspects of the disclosure may incorporate a liquid gate transistor, a variation of a field-effect transistor (FET), such as illustrated in FIG. 3.

Such FET-based sensors feature two essential electrodes: a source and a drain, facilitating the flow of charge carriers within the device. A critical component of such a FET-based sensor is a nanomaterial-based channel, which is responsible for modulating current. In such a design, the channel may be constituted of zinc oxide (ZnO) nanowires. ZnO nanowires exhibit remarkable properties that make them ideal candidates for biosensing applications, such as their high surface area and excellent electrical conductivity. The interaction between analytes and the ZnO nanowire channel results in changes in conductivity, enabling highly sensitive and specific detection of target molecules. The incorporation of ZnO nanowires into the transistor channel enhances the device's performance as a biological component detector, making it well-suited for various applications in the field of sensing and diagnostics. Source and drain electrodes are deposited on the thin films in such a way that the hybrid nanowires are placed perpendicular to the source and drain electrodes. The source electrode is connected to an external voltage or current supply, and the drain electrode is ground. The biological component detector is placed in a closed micro- or nanofluidic chamber for the supply of biological fluid to the biological component detector. The flow of the fluid inside the chamber can be controlled by an applied pressure or electric field. A fluid sample from cancer patients, such as pancreatic cancer and PDAC patients, can be directly injected into the fluidic chamber. Exosomes released from cancer cells, such as pancreatic cancer and PDAC cells, can be used as potential biomarkers for its early-stage diagnosis. Multiple ultrafiltration steps combined with ultracentrifugation is the preferred method, as it can give a maximum yield of the exosomes.⁵ For example, Glypican-1 (GPC-1) and MIF (macrophage migration inhibitory factor) are membrane-anchored proteins which are present in large quantities in exosomes released from PDAC cells.^{6,7} Binding of the GPC-1 and MIF with complementary antibodies present on the hybrid nanowires (such as functionalized semiconductor nanowires 200) can be recognized from multiple signals. The LSPR of noble metal nanoparticles in such hybrid nanowires is expected to show deviation in absorption maxima wavelength on the binding. This is due to a change in the local dielectric constant of the medium surrounding the noble metal nanoparticles. The molecular binding event can also be transduced in the form of a useful electrical signal which will be measured through real-time detection, or steady-state measurements based on the ionic strength of the sample. Recognition of the exosomes by the antibodies, present on the hybrid nanowires, changes the current or resistance across the nanostructure. The change can be detected by an external probe station connected to the biological component detector.

The optical measurements, in the context of a biological component detector device according to the disclosure, involve the use of light to assess changes in the behavior of noble metal nanoparticles (for example, silver and gold) due to the localized surface plasmon resonance (LSPR) effect. LSPR is a phenomenon where the electrons in the nanoparticles oscillate collectively in response to incident electromagnetic radiation, typically in the visible range of the electromagnetic spectrum. That resonance results in strong optical absorption and scattering properties that are highly sensitive to changes in the nanoparticle environment, making them valuable for biosensing applications. To measure the LSPR, before exposing a biological component detector according to the disclosure to cancer exosomes or any target analyte, an initial baseline measurement is taken. The baseline measurement serves as a reference point to capture the optical characteristics of the nanoparticles in their unbound state. The hybrid metal nanoparticle semiconducting nanowires (such as functionalized semiconductor nanowires 200) of the biological component detector is then exposed to a solution containing cancer exosomes, such as pancreatic cancer exosomes. In some instances, the antibodies on the hybrid metal nanoparticle semiconducting nanowire surface are specifically designed to bind with Glypican-1 (GPC-1) and MIF (macrophage migration inhibitory factor) present on the surface of pancreatic exosomes. As the pancreatic exosomes bind to the antibodies, they cause changes in the local refractive index around the nanoparticles of the hybrid metal nanoparticle semiconducting nanowire. That alters the LSPR conditions, leading to a shift in the resonance wavelength of the nanoparticles. The shift in resonance wavelength can be measured using optical spectroscopy techniques such as UV-Vis spectroscopy or surface plasmon resonance spectroscopy. In addition to wavelength shifts, changes in the intensity of the LSPR signal can also occur. The intensity of the scattered or absorbed light by the nanoparticles can increase or decrease, depending on the binding events. Those intensity changes are also monitored as part of the optical measurements. Any shifts in wavelength and changes in intensity are indicative of the presence and binding of pancreatic exosomes. Those alterations in the LSPR spectrum are specific to the interaction between the antibodies on the biological component detector's surface and the target analyte (pancreatic exosomes), demonstrating the sensor's ability to selectively detect the exosomes. The degree LSPR shift will depend on both the nanoparticle size and the surface coverage of biomarkers (antibodies) on the semiconductor nanowires. Larger nanoparticles and higher biomarker density lead to greater changes in the local refractive index, resulting in a more pronounced LSPR peak shift.

Extracting exosomes from pancreatic ductal adenocarcinoma (PDAC) samples typically involves a series of steps to isolate those small vesicles from the complex mixture of biological material. In some embodiments according to the present invention, the exosome extraction procedure is as follows:
1) Sample Collection and Preprocessing. PDAC tissue samples are homogenized in phosphate-buffered saline (PBS) and then subjected to low-speed centrifugation (e.g., 300× g for 10 minutes) to remove cells and debris.
2) Initial Ultra-Centrifugation. The supernatant obtained from the previous step is transferred to ultracentrifuge tubes and centrifuged at 10,000× g for 30 minutes at 4° C. to pellet larger microvesicles.
3) Filtration. The supernatant is passed through a 0.22 um filter to remove larger vesicles and debris.
4) Ultracentrifugation. The filtered supernatant is transferred to ultracentrifuge tubes and subjected to ultracentrifugation at 100,000× g or higher (typically 100,000-120,000× g) for 60-90 minutes at 4° C. This step pelts the exosomes at the bottom of the tubes.
5) Exosome Resuspension. The supernatant is gently removed without disturbing the pellet, and the exosome pellet is resuspended in a small volume of PBS. A protease and RNase inhibitor cocktail is used to protect the exosomes from degradation.

The description of the present embodiments of the invention has been presented for purposes of illustration but is not intended to be exhaustive or to limit the invention to the form disclosed. Many modifications and variations will be apparent to those of ordinary skill in the art. As such, while the present invention has been disclosed in connection with an embodiment thereof, it should be understood that other embodiments may fall within the spirit and scope of the invention. All patents and publications cited herein are incorporated by reference in their entirety.

### REFERENCES

(1) Turner, A. P. F. et al. “Biosensors: Sense and Sensibility,” Chem. Soc. Rev. 2013, 42(8), 3184-3196.

(2) Bellan, L. M. et al. “Current Trends in Nanobiosensor Technology,” Nanomed. Nanobiotechnol. 2011, 3(3), 229-24.

(3) Hooshmand, N. et al., “Collective multipole oscillations direct the plasmonic coupling at the nanojunction interfaces,” Proceedings of the National Academy of Sciences, 2019, 116(39), 19299-19304.

(4) Hooshmand, N. et al. “High-sensitivity molecular sensing using plasmonic nanocube chains in classical and quantum coupling regimes,” Nano Today, 2017, 17, 14-22.

(5) Yu, Z. et al., “Pancreatic Cancer-derived Exosomes Promote Tumor Metastasis and Liver Pre-metastatic Niche Formation,” Oncotarget, 2017, 8(38), 63461-63483.

(6) Melo, S. A. et al., “Glypican-1 Identifies Cancer Exosomes and Detects Early Pancreatic Cancer,” Nature, 2015, 523(7559), 177-182.

(7) Costa-Silva, B. et al., “Pancreatic Cancer Exosomes Initiate Pre-metastatic Niche Formation in the Liver,” Nat. Cell Biol., 2015, 17(6), 816-826.

What is claimed is:

1. A biological component detection system comprising:
a biological component detector comprising:
a source electrode;
a drain electrode; and
a plurality of functionalized semiconductor nanowires electrically coupling the source electrode and the drain electrode, each functionalized semiconductor nanowire comprising:
a semiconductor nanowire;
a plurality of nanoparticles dispersed on surfaces of the semiconductor nanowire; and
antibodies bound to surfaces of the semiconductor nanowire or the nanoparticles or a combination thereof; and
a microfluidic chamber configured for housing the biological component detector and delivering a biological component to the biological component detector.

2. The biological component detection system of claim 1, wherein the nanoparticles comprise or consist of a noble metal.

3. The biological component detection system of claim 2, wherein the noble metal is gold or silver.

4. The biological detection system of claim 1, wherein the semiconductor nanowire comprises silicon or zinc oxide.

5. The biological component detection system of claim 1, wherein the antibodies have a binding affinity for Glypican-1 (GPC-1), macrophage migration inhibitory factor (MIF), CA19-9, KRAS or a mutated KRAS, CD63, CD81, CD9, or carcinoembryonic antigen (CEA).

6. The biological component detection system of claim 1, wherein the functionalized semiconductor nanowires are placed on a substrate.

7. The biological component detection system of claim 6, wherein the substrate is made of one or more semiconductor materials.

8. The biological component detector of claim 1, wherein the biological component detector is a field effect transistor in the form of a liquid gate transistor.

9. The biological component detection system of claim 1, wherein
the biological component detector is configured to identify the biological component, and
the identity of the biological component is indicative of a cancer.

10. The biological component detection system of claim 9, wherein the biological component is an exosome or antigen of a cancer.

11. The biological component detection system of claim 9, wherein the biological component detector is configured to identify the biological component through UV-Vis spectroscopy.

12. The biological component detection system of claim 1, wherein the source electrode and drain electrode are made of gold or silver.

13. A method for evaluating the presence of a cancer within a subject, the method comprising:
obtaining a sample from the subject;
isolating biological components from the sample;
measuring the biological components using a biological component detection system according to a claim 1 wherein antibodies of the biological component detector bind to biological components; and
measuring a change in at least one of localized surface plasmon resonance (LSPR), conductivity, or electrical current of the biological component detector upon binding of the biological components to the antibodies.

14. The method of claim 13, wherein the subject is human.

15. The method of claim 13, wherein the biological components are indicative of a cancer.

16. The method of claim 13, wherein the sample is a blood sample, a stool sample, or a tissue sample.

17. The method of claim 13, wherein the change in LSPR is measured via UV-spectroscopy.

18. The method of claim 13, wherein the antibodies have a binding affinity for Glypican-1 (GPC-1), macrophage migration inhibitory factor (MIF), CA19-9, KRAS or a mutated KRAS, CD63, CD81, CD9, or carcinoembryonic antigen (CEA).

19. The method of claim 13. wherein the sample is processed prior to entering into the biological component detector.

20. The method of claim 19. wherein the sample is processed through at least one of liquification. homogenization, ultra-centrifugation, and filtration.
